(19) United States
(12) Patent Application Publication
(10) Pub. No.: US 2025/0195269 A1
(43) Pub. Date: Jun. 19, 2025

(54) HYDRO-CONTACT LENS ENABLING AN EYE TREATMENT DELIVERY SYSTEM AND METHOD

(71) Applicant: Miguelangel Salazar, Fort Myers, FL (US)

(72) Inventor: Miguelangel Salazar, Fort Myers, FL (US)

(21) Appl. No.: 19/072,492

(22) Filed: Mar. 6, 2025

Related U.S. Application Data
(63) Continuation-in-part of application No. 17/823,351, filed on Aug. 30, 2022, now abandoned.
(60) Provisional application No. 63/238,575, filed on Aug. 30, 2021.

(51) Int. Cl. A61F 9/00; G02C 7/04

(52) U.S. Cl. CPC A61F 9/0017; G02C 7/049

(57) ABSTRACT

An eye treatment method embodying an optical frame configured to form a watertight cavity around an eye of the patient, wherein the optical frame includes a corrective lens along a face thereof, and wherein the corrective lens is made from gas permeable material. The watertight cavity is selectively fillable with a treatment solution determined by the ailment and physical characteristics of the patient, the parameters of the corrective lens, and the properties of the gas permeable material.

### CROSS-REFERENCE TO RELATED APPLICATION

This application claims the benefit of priority of U.S. provisional application No. 63/238,575, filed 30 Aug. 2021, the contents of which are herein incorporated by reference. This application claims the benefit of priority of U.S. non-provisional application Ser. No. 17/823,351, filed 30 Aug. 2022, as a Continuation In Part, the contents of which are herein incorporated by reference.

### BACKGROUND OF THE INVENTION

The present invention relates to ophthalmic lenses and, more particularly, an optical device for hydration of the eyeball, wherein the optical device includes a gas permeable corrective screen fixed to a frame, wherein the corrective screen is dimensioned and adapted to adhere to the eye socket to form a liquid chamber so that, when filled with isotonic solution, becomes a liquid body contacting the entirety of the exposed eye and thereby correcting atypical ametropia, which is impossible for conventional ophthalmic lenses.

The most frequent ametropias include myopia, hyperopia, astigmatism, presbyopia, irregular astigmatism, irregularity after refractive surgery or corneal implants with remaining distortion of corneal curvature, keratoconus, marginal degeneration, and corneal trauma that leave scars. Other important conditions include dry eye syndrome, low vision, strabismus, phorias, and marked anisometropia.

The only existing device that claims to address the problem of dry eye resulting from the lack of proper hydration of the eyeball is the corneal scleral device (scleral support contact lens), putatively providing corrective vision. However, it is not entirely true, since the hydration it generates is limited to the diameter of the corrective lens that directly engages the eye, the standard scleral support lens having a diameter of approximately 25 millimeters. Thus, it is not correct to say that the corneal scleral support device dominates the dry eye syndrome. On the other hand, the correction of atypical ametropia, until now, has been the exclusive use of rigid and specialized contact lenses.

Existing devices or systems (specifically, the use of contact lenses) do not work 100% in the case of dry eye syndrome. Specifically, the scleral support contact lens only provides hydration within its circumferential bounds, and thus the portion of the wearer's eye outside that circumference is untreated. Furthermore, contact lenses, of any type, cannot be used in cases of infections or ocular affections, as well as in cases where the patient must undergo specialized studies. Similarly, contact lenses do not work well in cases where the patient needs to rest his eyes, after a reasonable time of use. Likewise, contact lenses do not work well in cases where the patient, due to a disability or simply because he/she does not have the required dexterity due to some factor that prevents it (e.g., nerves, restlessness), is not capable of inserting the contact lenses by him/herself.

As can be seen, there is a need for an optical device for hydration of the entirety of the exposed eyeball, wherein the optical device includes a gas permeable corrective screen fixed to a frame, wherein the corrective screen is dimensioned and adapted to adhere to the eye socket so as to define a substantially hermetically sealed liquid chamber therearound that, when filled with isotonic solution, becomes a liquid volume that envelopes the entirety of the exposed eye, correcting dehydration-related ailments of the eye along an entirety of the exposure portions of the eye. The hydro-contact lens apparatus embodied in the present invention can correct refractive ametropia, as well as conventional and atypical vision, through completely hydration of the anterior segment of the eyeball up to its attachments, without any diameter limitation. It can also be used for ophthalmic medication treatments that require exposure for specific times without loss of dose. All these problems are addressed with high performance. Likewise, when there are infections or bacterial eye conditions in the eye, the first thing that is recommended to the patient is that he/she cannot use contact lenses during the ophthalmic treatment. The hydro-contact spectacle solves this problem, since the patient can use it during the treatment and maintain the vision, since the hydro-contact spectacle/goggle is not in contact with the anterior segment of the eyeball and so it can be kept in use during the corresponding medical treatment and provide the necessary vision to the patient. Finally, in the case of patients with atypical ametropia who must undergo specialized studies, for example, corneal topography, where they are prevented from using contact lenses and are considered disabled, the hydro-contact spectacle is the solution to the problem, since contact lenses cannot be used either before or during these studies.

### SUMMARY OF THE INVENTION

The hydro-contact optical device embodied in the present invention is a visual correction device, which differs in its structure, mainly because it has no physical contact with the cornea, and only water contact on the surface of the anterior segment of the eyeball, which makes the invention unique, novel and different from everything that exists and, together with the other existing devices, the hydro-contact eyeglass is the best alternative for patients who require it, especially for the most visually impaired.

In the case of atypical ametropias, the present invention becomes a lens that corrects and resolves irregular refractions, thus generating a coherent refraction, being molded against the inner face of the corrective shield and being contained in front of the cornea, it becomes a second lens, and this liquid containment neutralizes the irregular refraction, allowing the optical correction of all the aforementioned visual defects to be resolved.

Likewise, in these cases, the hydro-contact spectacle component, called the corrective shield, has the capacity to generate the necessary diopters, this is achieved by its internal base curvature and by its corrective carving on the posterior face.

As for other important conditions, the hydro-contact spectacle lens solves them as follows: (i) the liquid interaction, provides permanent hydration to the eyeball for the case of dry eye syndrome, of any scale, (ii) the focal distance in front of the cornea can be varied in case certain magnifications condition, such as in cases of low vision, and (iii) as for the dioptric power correction, it is handled by making variations of the base curve of the corrective shield and, additionally, with the dioptric power that is calculated to the corrective shield.

In the case of treatments with ophthalmic medication, the present invention enables medication to be diluted in the liquid chamber so that it has an interaction with the anterior segment of the eyeball, and since the prescribed medication is contained in the fluidly sealed liquid chamber, there is no possibility of drainage, thus the patient could achieve an optimal recovery.

The hydro-contact goggle solves and selectively attends the optical dysfunctions or syndromes, such as dry eye syndrome. In cases where treatment or study of the eye is required, the present invention allows patients to have a device within their reach that provides vision during the time of the treatment and/or study. The present invention is an optical device that works correctly. In short, the hydro-contact spectacle solves cases where the patient, for the reasons described above, cannot wear contact lenses.

The hydro-contact spectacle solves the dry eye syndrome problem through completely hydrating the entirety of anterior segment of the eyeball up to its attachments, without any diameter limitation.

Now, with the present invention of the hydro-contact spectacle, not only is a new field being born, but it also has the ability to resolve ametropia without the sensation of a foreign body. Likewise, when there are infections or bacterial conditions in the eye, the first thing that is recommended to the patient is that he/she cannot use contact lenses during the ophthalmic treatment. The hydro-contact spectacle obviates this restriction, as the patient can use it during the treatment and also maintain the vision, since the hydro-contact spectacle is not in contact with the anterior segment of the eyeball.

In one aspect of the present invention, a goggle or goggles can be configured to fit over one or both eyes of a patient and seal against the skin, around the eye sockets. The goggle or goggles can be sufficiently airtight to allow a desired air pressure to be maintained in one or more cavities or liquid chambers inside the goggle or goggles and over the eye(s) and hermetically sealed from the external environment by said airtight connection around the eye socket(s). There can be a seal material 121 positioned around a perimeter of the goggle frame, which can create a seal between edges of the goggle or goggles and the patient's skin around the eyes.

In another aspect of the present invention, an eye treatment device includes the following: a goggle frame, having at least one cavity, configured to surround and be spaced from an eye; a corrective lens along a face of the goggle frame; and an inlet formed in an upper portion of the goggle frame, the inlet fluidly communicating the cavity to an external environment; further including: a sealing material along a perimeter of the goggle frame for forming a hermetical seal against skin around a socket of the eye, wherein the sealing material is a portion of a suction cup; and further including a tapered stopper for fluidly sealing the inlet, wherein the corrective lens is made from gas permeable material having a thickness of approximately 0.70 millimeters, wherein a distance between a distance between a back surface of the corrective lens and a front of a of the eye is selected as function of a radius of curvature of the corrective lens.

In yet another aspect of the present invention a method of treating dry eye syndrome, the method includes providing the above-mentioned eye treatment device and filling the cavity with a fluid by way of the inlet.

In an aspect of the subject disclosure a method of treating a medical condition of a patient with an immersive solution having a medicament to remedy the medical condition includes the following: assessing a set of ocular properties of the patient; sealing a cavity around an eye of the patient by way of a frame with a lens holder disposed in a forward-facing element of the frame, wherein the lens holder retains a first lens having a first radius of curvature; filling the cavity with an isotonic solution; re-assessing the set of ocular properties of the patient with the first lens and the isotonic solution disposed in a field of view of the patient; determining a second lens with a second radius of curvature; and determining a percentage or weight of the medicament to be mixed with the isotonic solution to form the immersive solution within the cavity, based on the second radius of curvature and the re-assessed set of ocular properties.

In another aspect of the subject disclosure, a method of treating an ocular condition that adversely affects the vision of a patient includes the following: sealing a mounting frame against the patient so as to define a sealed chamber that circumscribes an eye of the patient, wherein the mounting frame provides a corrective lens for correcting the vision of said eye; determining a vertex distance, a base curve, and the gas permeability for the corrective lens based on a diopter of the eye; selectively filling the chamber with a medical solution based on a treatment plan for the ocular condition, wherein the treatment plan includes a measurement of the frame.

These and other features, aspects and advantages of the present invention will become better understood with reference to the following drawings, description, and claims.

### BRIEF DESCRIPTION OF THE DRAWINGS

FIG. 1 is a flow chart of an exemplary embodiment of the subject disclosure regarding ocular treatment systems and methodologies.

FIGS. 2A-2D are a cumulative series of flow charts for embodiments of the subject disclosure as it pertains to the ocular treatment systems and methods.

### DETAILED DESCRIPTION OF THE INVENTION

The following detailed description is of the best currently contemplated modes of carrying out exemplary embodiments of the invention. The description is not to be taken in a limiting sense but is made merely for the purpose of illustrating the general principles of the invention, since the scope of the invention is best defined by the appended claims.

Broadly, the present invention includes an eye treatment device includes the following: a goggle frame 2, having at least one cavity, configured to surround and be spaced from an eye; a corrective lens along a face 22 of the goggle frame; and an inlet formed in an upper portion of the goggle frame, the inlet fluidly communicating the cavity to an external environment; further including: a sealing material 54 along a perimeter of the goggle frame for forming a hermetical seal against skin around a socket of the eye, wherein the sealing material is a portion of a suction cup; and further including a tapered stopper for fluidly sealing the inlet, wherein the corrective lens is made from gas permeable material having a thickness of approximately 0.70 millimeters, wherein a distance between a distance between a back surface of the corrective lens and a front of a of the eye is selected as function of a radius of curvature of the corrective lens.

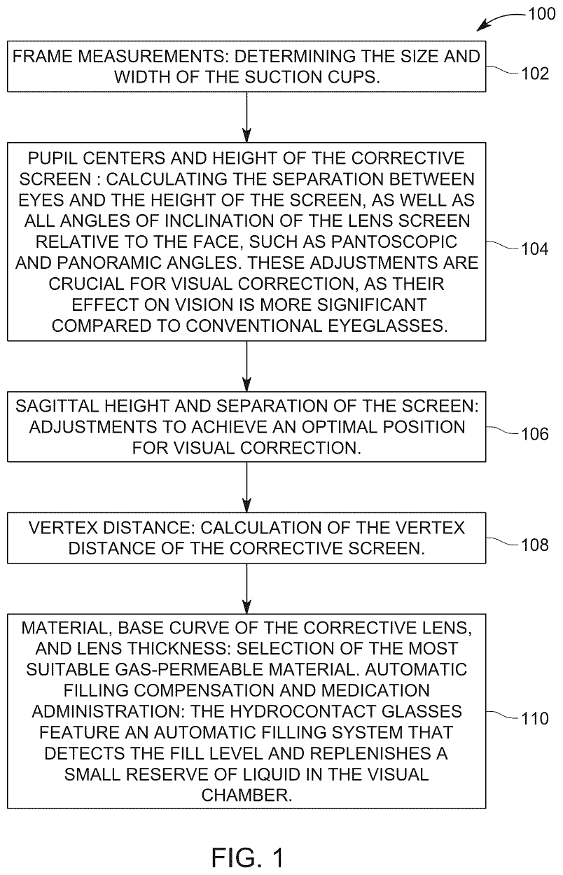

Referring now to FIGS. 1 and 12, the hydro-contact lens assembly 100 of the present invention may include one or two gas permeable corrective screens 1, each corrective screen 1 dimensioned and adapted to correct diopters. Each gas permeable corrective screen 1 has a form and shape determined by the carving of the material on its external face, as well as by the water exchange of its internal base curvature. This material may vary in diameter and permeability. Each gas permeable corrective screen 1 has a thickness of approximately 0.70 millimeters. Each gas permeable corrective screen 1 is made from material, such as polycarbonate material, providing ultraviolet protection. Each gas permeable corrective screen 1 is removable for cleaning.

The hydro-contact lens assembly 100 may include a separate frame 2 to which each gas permeable corrective screen 1 is fixed by way of a rubber gasket 8. The rubber gasket 8 may be dimensioned and adapted to hermetically or fluidly fixing the corrective screen assembly 1 on the frame 2. It also hermetically seals the liquid contents within the liquid chamber formed anteriorly around the eyeball of the human user. The rubber gasket 8 may be made of one hundred percent (100%) silicone material.

Each frame 2 may provide one or more auxiliary inputs/inlets 3. Each auxiliary inputs 3 is a filling port located on the upper outer edge of the frame 2, with respect to each eye socket. The function of these inlets 3 is to enable practitioners to urge the solution or doses of medication (e.g., during ophthalmic medical treatments), thus ensuring that they are not lost at the time of placement.

A closing plug 4 may be provided to close (plug) the auxiliary inlet 3. The closing plugs tapered geometry facilitates a fluid seal forming at the inlet 3. The closing plug 4 may be one hundred percent (100%) silicone material and conform to the plateau.

A suction cup 5 may be connected to the frame 2 proximal the gas permeable corrective screen assembly 1. Both the suction cup 5 and the frame 2 may provide complementary channels 58 and 28, respectively, for facilitating the interface of the suction cup 5 to the frame 2. The channel 58 of the suction cup 5 is provided by an interfacing portion 56.

The suction cup 5 is dimensioned and adapted to adhere to the skin adjacent to the eye sockets and under pressure maintains the seal to define a liquid chamber 10 anterior the eyeball of the human user, wherein liquid is kept inside the liquid chamber 10 without liquid leakage occurring. The use of suction cups 5 avoids exposure to chemical residues resulting from the use of adhesives for adhesion to the skin. The suction cups 5 for the hydro-contact lens assembly 100 will be designed for liquid containment, and the skirts 52 of the suction cups adhere to the facial surface, both internally and externally, very different from the design of diving or swimming masks.

A bridge 6 may join two frames 2 and may be made of one hundred percent (100%) silicone material and must be elastic. It is understood that the hydro-contact lens assembly 100 contemplates the use of only one corrective lens 1 and cooperating frame 2, analogous to an eye patch embodiment.

A strap 7 engages the opposing frames 2 for selectively adjusting around the pressurized head to keep the liquid chamber 10 in the correct position. The strap 7 may be made of one hundred percent (100%) silicone material and must be elastic.

The relationship between the corrective shield 1 and its mounting frame 2 contemplates the vertex distance (the distance between the back surface of a corrective lens 1 and the front of the cornea) and the presence of fluid therebetween influencing the dioptric correction. This is directly related to the rubber gaskets, which directly influence the calculation of the angles and distances in front of the eyeball, which allows the support and the liquid content within the liquid chamber 10. These three components are directly related to vision or visual function, and the liquid content to the refraction of light. The relationship of the auxiliary inlets 3 with the frame is to allow the liquid to be poured in such a way that it does not spill, it is recommended that this be done in a vertical position, to achieve the most optimal liquid containment. The relationship of the plug and the auxiliary inlets is to pressurize and depressurize the hydro-contact eyeglass from the atmospheric pressure against the liquid content, releasing the atmospheric pressure of the instrument adhered to the eye at the moment of removing it, even avoiding a liquid drainage or liquid precipitation towards the nasolacrimal duct (explained by the Boyle-Mariotte Law), thus generating more guarantees for the visual health.

Likewise, the stopper 4 prevents the entry of external agents that may contaminate the solution, such as: mites, dust, bacteria, viruses, foreign bodies and natural evaporation, thus avoiding a higher saline concentration (where the liquid content becomes denser). The cap also prevents, in the event of an impact, the generation of atmospheric shock pressure when the goggle is removed. The plug 4 has a diameter and adjustment ratio in relation to the auxiliary inlet 3, since it has an adjustable base that hermetically seals the liquid containment and is easily released by turning or direct insertion. The bridge is interchangeable and is directly related to the frame, since it allows the frame to be anchored.

The bridge 6 collaborates in the adjustment of the shields and suction cups 5, in a more anatomical position of adhesion to the ocular cavity. Likewise, the bridge 6 enables effective and decisive adjustment due to its interchangeability capacity (change of sizes) for making selectively adjustments in view of the interpupillary distance; however, these calculations are made previously before assembly, but there is a freedom of adjustment with the bridge 6 that enables such selective adjustment. Also, the bridge 6 has a direct relationship with the suction cups 5 and the frame 2, making the suction cups 5 mold to the facial vault in a strong way, given the fact that they are anchored to the frame 2. The straps 7 are related to the anchor and the frame and are responsible for the compression required to achieve liquid containment through the suction cups 5 and facial vault or cavity defined by the frame 2 and possibly the suction cup 5 that seals against the skin, around the eye sockets.

Facial cavity/liquid chamber 10 bonding connection by way of sealing material (in certain embodiment, the material along the periphery of a skirt 52 of the suction cup 5). Relatedly, the skirts 52 that are projected at an outward and inward angle. The design geometry allows the liquid containment space to be reduced in a more usable way and avoids a displacement zone of liquid leakage.

The present invention embodies a method of fixing the corrective screen component on the frame base and on the fixing rubber seals. The mounting of the corrective screen can be adjusted to the distance to the vertex. It is required to calculate the panoramic angle, pantoscopic angle and facial angle prior to mounting, since it is not advisable to adjust afterwards, as this instrument contains liquid, and any possibility of solution spillage must be avoided.

A liquid chamber 10 is the product of the process of properly wearing the hydro-contact lens assembly 100. The liquid chamber's function is to contain the liquid and it is the corrective medium. This structure is like a screen of an atmospheric isolation telescope.

One of many filling systems are contemplated by the present invention as long as liquid is urged into the liquid cavity, be it gravity-urged through the filler neck or otherwise. It is recommended to tilt the head slightly backwards to avoid air spaces, then each inlet is closed. This process does not generate an additional variant of ocular pressure.

The sealing plug 4 functions to close (plug) the auxiliary inputs 3. The sealing plug 4 is easy to locate by touch and canalizes the solution filling due to its geometry.

Distance to the corrective screen vertex: the distance to the vertex will generally be as close to the eye as possible (without having physical contact with the eyelids) can be adjusted by tightening and loosening of the strap 7.

A method of making the present invention may include the following. A manufacture would understand that the hydro-contact lens assembly 100 is not for generic use, and so must be custom made for each person depending on their visual condition, and only a technician specialized in optometry and contact lens, with the given specifications of the invention, optical health management, training and optometric calculations, could adapt it.

The material used for the permeable corrective screen assembly 1 is a critical element, but it can also be improved by increasing the permeability with the use of another material that will improve the existing oxygen transmissibility.

The only way to be able to shuffle, exchange or reconfigure some element or component of the invention, would be to improve the initial design of the prototype, that goes more in line with the anatomy, as well as at the manufacturing level, to be able to have access to materials of permeable characteristics of larger diameters for the corrective screen 1 that allows a larger field of view. The present invention contemplates the structure or frame 2 to be attached to the facial vault or eyelids without using the components of the suction cup, bridge, and strap as they are thought in the initial prototype.

A method of suing the present invention may include the following. First, the user may disassemble the corrective shields for cleaning and wash them thoroughly with products recommended for the contact lens industry for gas permeable materials. Second, the user must wash the structure or frame very well, preferably with water and hypoallergenic soap. Third, the user rinses with plenty of water, both the corrective screens and the frame (steps one and two). Fourth, the user may remove the sealing plugs. Fifth, the user may install the corrective screens on the structure. Sixth, the user may place the assembled structure around the head in the primary gaze position. It is very important that the user removes the closing caps beforehand. Seventh, the user may adjust the suction cups around the eye sockets, fastening correctly by means of an elastic strap to the head, with sufficient pressure so that there is no leakage during liquid filling. Eighth, the user may pour the isotonic solution from the upper part through the auxiliary inlets, once the liquid chamber 10 is full, it is recommended to wait a few minutes (subject to clinical studies) for further thermal compensation of the solution and close the filling inlets with the closing plugs. Ninth, once the device is in place, the user may verify that it is filled with the solution and that it is securely fastened. If necessary, the user may have to tilt the head slightly backwards or sideways to level the filling of the solution. Tenth, the times of use will be at the discretion of the physician by subsequent clinical studies, which will determine the most appropriate recommendations. Eleventh, before removing the hydro-contact lens assembly 100's seal material/suction cup 5, each auxiliary inlet must be opened to release the pressure before removal.

Additionally, the present invention was devised for the field of visual health, originally aimed at correcting atypical visual defects, as well as affections of the visual organ. It is feasible that its elements or parts can be fabricated through the direction of a computer with the appropriate technology.

Furthermore, immersive administration of ophthalmic medication delivery methods that use the hydro-contact lens system embodied in the subject disclosure exceeds the corrective visual therapy capabilities offered by the prior art.

As can be seen, there is a need for treating ocular conditions through the combination of a transmissive optical device (e.g., corrective lens) and immersive administration of medical solutions to the entirety of the exposed eyeball. The hydro-contact lens system of the subject disclosure provides a frame removably sealable to the face of the patient for forming a chamber circumscribing the patient's eye, wherein the chamber is selectively fillable with medical solutions for immersive administration to the exposed eye. The frame also includes a gas permeable corrective screen adjustably distanced from the exposed eye. The hermetically sealed liquid chamber surrounds the exposed eye, so that when filled with medicinal solution, the exposed eye is immersed in the medicinal solution. Thus, the subject disclosure corrects dehydration-related ailments and administers immersive medicinal solution to the eye, all while facilitating vision through the transmissive optical lens.

Within the liquid chamber/cavity around the eye of the user, with the chamber featuring a gas-permeable interchangeable optical device, immersive administration of medicinal formulations enable a revolutionary tool that combines the comfort of ocular hydration with precise visual correction.

Furthermore, the device can be manufactured as a corrective patch or disposable visual correction dressing, affixed to the facial vault using adhering suction cups, eliminating the need for an elastic strap. The corrective patch provides a less complex design that focuses on ocular treatments and severe dry eye syndrome, as well as special visual conditions. Such visual conditions include irregular astigmatism, such as in cases of corneal scarring and keratoconus, where the cornea loses its geometric symmetry and conventional glasses are ineffective. Previously, only contact lenses could be used, but now, with the “hydro-contact glasses” enabled by the optical device and immersive fluid formulation delivery system, the subject disclosure functions both as glasses and as contact lenses, allowing visual correction through optical interaction with the treatment formulation in the liquid chamber and the gas-permeable lens screen.

Features and functioning of the treatment delivery system and method embodying the hydro-contact lens with corrective formula function (either as a visual patch or glasses) that fit comfortably around the eye socket include, but are not limited to, the following:
1. Customized corrective formularization for delivering a tailor medicinal formulation adapted to the visual correction needs of each user, including common and atypical ametropias.
2. Comfort and hydration through complete immersion around the eye, keeping the ocular surface fresh and comfortable, reducing eye strain during the medicinal drug delivery process.
3. Versatile visual patch adapted to be used in various situations, such as temporary visual correction or visual rehabilitation therapy.

The benefits and applications of the subject disclosure include tailormade visual correction through the combination of the customized corrective formula and ocular hydration ensures effective visual correction.

The subject disclosure also affords therapeutic versatility as it is usable in the treatment of visual disorders, providing personalized support and correction. Comfort and safety are enabled as the configuration of the design avoids direct contact with the cornea, offering a comfortable and safe visual experience.

In sum, the hydro-contact optical device in combination with corrective formula represents a significant evolution in ocular health, combining hydration and personalized visual correction.

This subject disclosure stands out as an effective tool in vision care, offering additional benefits to users, including but not limited to integration of smart technology including the following:
1. Integrated Sensors configured to constantly monitor temperatures and liquid fill levels, as well as oxygenation, ensuring an optimal ocular environment.
2. Wireless connectivity and synchronization with mobile applications for remote adjustments and personalized notifications are contemplated herein.
3. Automatic adjustment of visual correction through automatic calibration according to the changing needs of the user. The motorized capability in the mounting ring allows for orientation of positions in the lens to modify its separation distance from the eyeball of the patient, making changes in visual correction through the focal distance of the lenses in front of the eye.
4. Personalized notifications, wherein the optical device is configured to send reminders about changes in the solution and necessary adjustments in the position of the lenses within the structure.
5. Data analysis and monitoring capabilities, wherein the sensors are configured to collect data on device use and ocular health.

By way of example, a method to correct visual defects such as keratoconus 100 involves a specialist performing a detailed assessment. The steps of the corrective method 100 include, but are not limited to, the following:

Step 102 includes frame measurements determining the size and width of the suction cups.

Step 104 involves locating the pupil centers and height of the corrective screen, which involves calculating the separation between eyes and the height of the screen, as well as all angles of inclination of the lens screen relative to the face, such as pantoscopic tilt and panoramic angles. These adjustments are crucial for visual correction, as their effect on vision is more significant compared to conventional eyeglasses.

Step 106 includes determination of the sagittal height and separation of the corrective screen or lens, which contemplates adjustments to achieve an optimal position for visual correction.

Step 108 includes calculations of the vertex distance of the corrective screen/lens.

Step 110 involves selection of the material, base curve of the corrective lens, and lens thickness. Selection of the most suitable gas-permeable material for the hydro-contact lens of the subject disclosure features an automatic filling system that detects the fill level and replenishes a small reserve of the liquid (treatment formulation) in the visual chamber for medication administration. Additionally, the subject disclosure allows for controlled medication administration under medical supervision, enhancing the treatment's effectiveness.

The subject disclosure increases treatment efficacy through direct medication administration at localized areas of the eye, where medical control and supervision are facilitated by the subject disclosure to optimize therapeutic outcomes through controlled dosing. The subject disclosure provides a comprehensive solution for ocular care. The automatic filling compensation capability and medication administration of corrective formula by way for the liquid chamber sealed about the exposed eye of the patient represents a significant advancement in combining visual correction, ocular hydration, and personalized ophthalmological treatment. This innovation not only enhances treatment efficacy but also provides a comprehensive and safe solution for various visual and therapeutic needs.

The development of the hydro-contact lens optical device with corrective formula, along with the incorporation of smart technology and medication administration capabilities, sets a new standard in ocular health care.

This device not only addresses visual correction issues but also offers a personalized and comfortable experience for the user. With the integration of advanced monitoring and adjustment systems, the glasses become a versatile tool that adapts to the changing needs of patients, optimizing their visual and overall health.

This innovative approach promises to transform the way visual conditions and disorders are treated and managed, providing users with a higher quality of life and more effective access to advanced ophthalmological solutions. The combination of comfort, functionality, and technology positions the hydro-contact lens with corrective formula at the forefront of future ocular care.

The utilization and adaptation of hydro-contact lens with a corrective formula is a comprehensive process that requires the expertise of a visual health professional, such as an optometrist or ophthalmologist, who is certified in the adaptation of this device. This process not only focuses on precise measurement but also evaluates the individual needs of the patient.

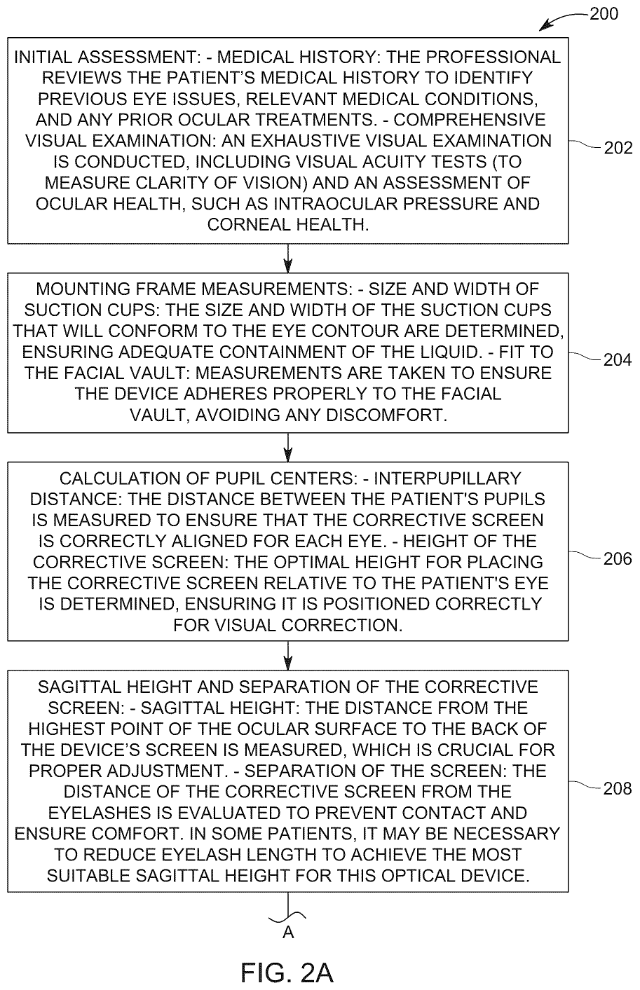

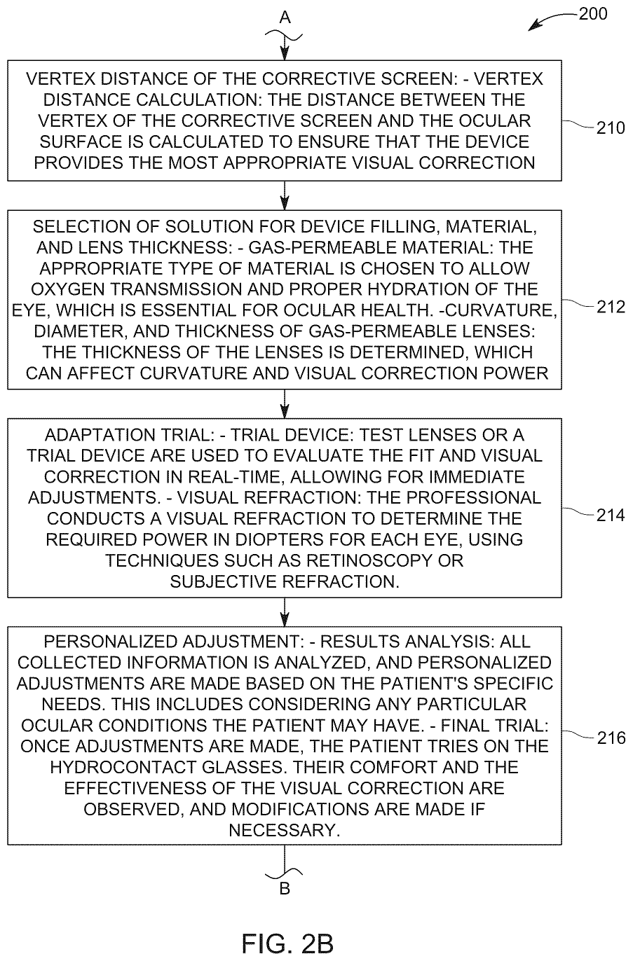

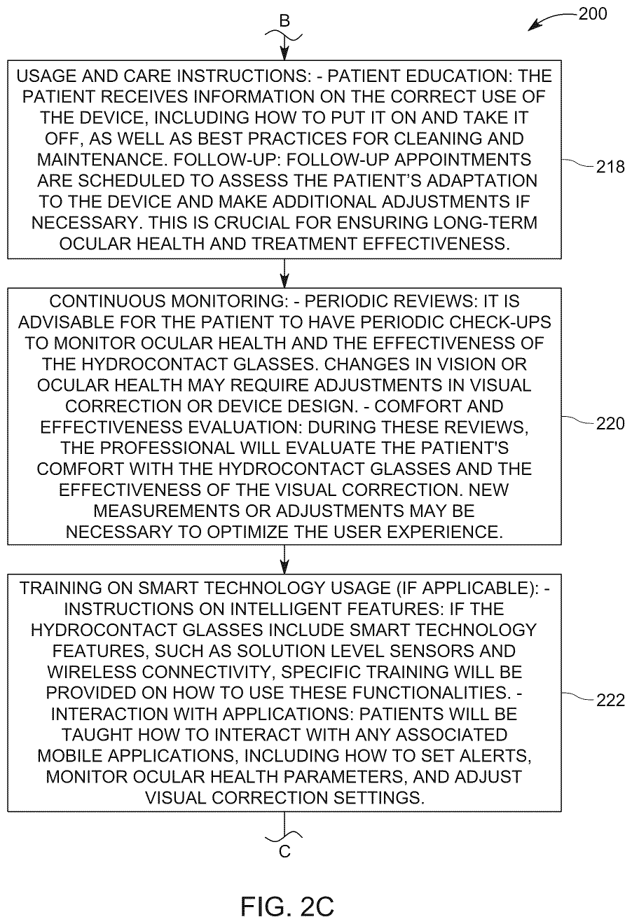

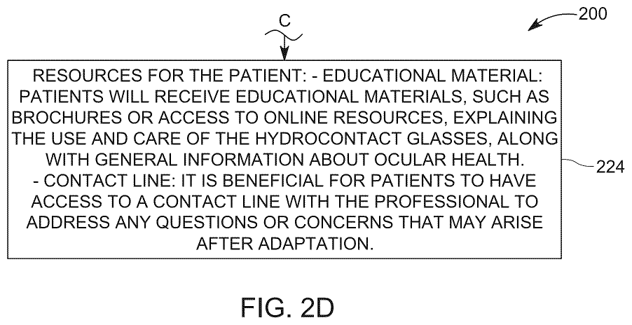

Referring to FIGS. 2A through 2D, the subject disclosure embodies the following detailed steps or method ocular treatment 200:
1. Initial assessment step 202, where the professional reviews the patient's medical history to identify previous eye issues, relevant medical conditions, and any prior ocular treatments. The professional provides a comprehensive and exhaustive visual examination conducted, including visual acuity tests (to measure clarity of vision) and an assessment of ocular health, such as intraocular pressure and corneal health.
2. Mounting frame measurements follow, where the size and width of the suction cups (step 204) that will conform to the eye contour are determined, ensuring adequate containment of the liquid. Measurements are taken to ensure the cavity defining portion of the device snugly fits and adheres properly to the facial vault, avoiding any discomfort.
3. Calculation of pupil centers and interpupillary distance (step 206), wherein the distance between the patient's pupils is measured to ensure that the corrective screen is correctly aligned for each eye. The optimal height for placing the corrective screen or lens relative to the patient's eye is determined, ensuring it is positioned correctly for visual correction.
4. Sagittal height and separation of the corrective screen determination (step 208), where the sagittal height is the distance from the highest point of the ocular surface to the back of the device's screen is measured, which is crucial for proper adjustment. Separation of the screen, i.e., the distance of the corrective screen from the eyelashes is evaluated to prevent contact and ensure comfort. In some patients, it may be necessary to reduce eyelash length to achieve the most suitable sagittal height for this optical device.
5. In step 210, the distance between the vertex of the corrective screen and the ocular surface is calculated to ensure that the device provides the most appropriate visual correction.
6. Selection of solutions for device filling, material, and lens thickness of the—gas-permeable material-step 212—the appropriate type of material is chosen to allow oxygen transmission and proper hydration of the eye, which is essential for ocular health. The curvature, diameter, and thickness of gas-permeable lenses are determined, which can affect curvature and visual correction power.
7. In an adaptation trial, step 214, test lenses or a trial device are used to evaluate fit and visual correction in real-time, allowing for immediate adjustments. The professional conducts a visual refraction to determine the required power in diopters for each eye, using techniques such as retinoscopy or subjective refraction.
8. A personalized adjustment (step 214) where all collected information is analyzed, and personalized adjustments are made based on the patient's specific needs, which includes considering any ocular conditions the patient may have. This includes a final trial: once adjustments are made, the patient tries on the hydro-contact lens. Their comfort and the effectiveness of the visual correction are observed, and modifications are made if necessary.
9. Usage and care instructions and patient education, step 218, includes the patient receives information on the correct use of the device, including how to put it on and take it off, as well as best practices for cleaning and maintenance. Follow-up appointments are scheduled to assess the patient's adaptation to the device and make additional adjustments if necessary. This is crucial for ensuring long-term ocular health and treatment effectiveness.
10. Continuous monitoring and periodic reviews make up step 220. It is advisable for the patient to have periodic check-ups to monitor ocular health and the effectiveness of the hydro-contact glasses. Changes in vision or ocular health may require adjustments in visual correction or device design. Comfort and effectiveness evaluations include professional reviews that evaluate the patient's comfort with the hydro-contact glasses and the effectiveness of the visual correction. New measurements or adjustments may be necessary to optimize the user experience.
11. Training in smart technology usage (if applicable) forms step 222. If the hydro-contact lens includes smart technology features, such as solution level sensors and wireless connectivity, specific training will be provided on how to use these functionalities. Patients will be taught how to interact with any associated mobile applications, including how to set alerts, monitor ocular health parameters, and adjust visual correction settings.
12. Resources for the patient include educational material, defining step 224. Patients will receive educational materials, such as brochures or access to online resources, explaining the use and care of the hydro-contact glasses, along with general information about ocular health. It is beneficial for patients to have access to a contact line with the professional to address any questions or concerns that may arise after adaptation.

The measurement and adaptation process for hydro-contact glasses with corrective formula is a complex procedure that requires the active involvement of a visual health professional. This process ensures that the device fits precisely to the individual needs of the patient, optimizing visual correction and comfort.

Continuous attention and follow-up are essential to guarantee that the glasses remain effective and safe over time. The combination of advanced technology, personalization, and professional care allows hydro-contact glasses to offer an innovative and effective solution for correcting visual problems, thus improving the patient's quality of life.

Ultimately, the success in adapting this type of device depends not only on precise measurements but also on effective communication between the professional and the patient, as well as the patient's commitment to their ocular care.

This comprehensive approach to the adaptation and ongoing management of hydro-contact glasses with corrective formula positions them as a pioneering solution in the field of ocular health. By addressing both the functional and therapeutic aspects of visual correction, these glasses offer a promising alternative for individuals with specific visual needs, enhancing their everyday lives and providing a pathway to improved ocular health.

Furthermore, the hydro contact goggle embodied in the subject disclosure provides an FDA-approvable solution for treating corneal hypoxia (lack of oxygen to the cornea) as well as other ocular conditions and ailments to the eyes.

When the cornea (which is the part of the eye that helps you see) doesn't get enough oxygen, some of the consequences:
1. Red Eyes: The cornea can become irritated, making the eyes red.
2. Blurred Vision: It may be difficult to see clearly.
3. Discomfort: The eyes may hurt or feel uncomfortable.
4. Swelling: The cornea may swell and become slightly opaque, which also affects vision.
5. Infections: If you do not have enough oxygen, it may be easier for the eyes to become infected.
6. Corneal changes: Lack of oxygen can damage the cornea over time.

Anything worn over one's eyes, such as contact lenses, allows oxygen to pass through, so our eyes can stay healthy, since it is important to ensure an adequate supply of oxygen to the cornea, especially in the context of medical products and optical devices, such as contact lenses. Eye health is critical not only to individual well-being, but also to overall quality of life. Here are several key points to consider:
1. Patient Health and Safety: The cornea, being avascular, depends on the oxygen it gets from air and tears. Lack of oxygen can cause serious complications, including inflammation, infection and permanent vision damage. Manufacturers of medical devices, including contact lenses, have a legal responsibility to ensure that their products do not compromise the health of the wearer.
2. FDA Regulations: The Food and Drug Administration (FDA) regulates medical devices, so any product that is used in or on the eye must meet strict standards for safety and efficacy. Lenses that do not allow oxygen to pass through may not be approved, as their use could result in damage to eye health, posing a significant risk to consumers.
3. Manufacturer's Liability: If a product causes damage due to its design or lack of adequate information on its use, the manufacturer may be held legally liable. This underscores the importance of contact lenses and other optical devices being designed to allow oxygen permeability, thus avoiding eye injuries and potential negligence claims.
4. Consumer Education: It is critical that consumers are informed about the importance of using oxygen-permeable products. Lack of education can lead to improper use of unsafe eyewear, which can result in health problems that could have been avoided.
5. Innovative Product Development: The optical industry has a responsibility to innovate and develop products that not only comply with regulations but also prioritize eye health. Investing in research and development of safe and effective materials is essential for the advancement of eye health.

In short, ensuring that the cornea receives sufficient oxygen is of paramount importance not only for the individual health of patients, but also for compliance with legal regulations and the social responsibility of manufacturers. Eye health must be a priority, and every stakeholder, from manufacturers to healthcare professionals and consumers, has a role to play at promoting and protecting it.

The difference between a recreational and a medical lens regarding oxygen permeability can be summarized in several key aspects:

### 1. Purpose and Function:

Recreational Lenses: generally, they are designed for activities such as swimming, sports or eye protection. Their primary function may be vision correction or simply protection from water, sun or dust. Oxygen permeability may not be a top priority, as their use is usually temporary.
Medical Lenses: These are specifically designed to treat or correct vision problems and, in many cases, can be worn for extended periods of time. The health of the cornea is crucial to the success of treatment, so oxygen permeability is essential to avoid complications.

### 2. Regulations and Standards:

Recreational Lenses: they may be subject to less stringent regulations compared to medical devices. Regulations may focus mainly on general safety and protection.
Medical Lenses: they are regulated by the FDA and must meet strict standards for safety and efficacy, including the ability to allow oxygen to pass to the cornea to prevent eye problems.

### 3. Materials Used:

Recreational Lenses: they may be made by materials that are not necessarily designed to be oxygen permeable, as their use is not permanent, and they are not expected to be in direct contact with the surface of the eye for extended periods of time.
Medical Lenses: These use advanced materials that aim to high oxygen permeability, ensuring that the cornea receives the necessary oxygen for its health, especially during prolonged wear.

### 4 Duration of Use:

Recreational Lenses: they are usually used for short periods, such as during a specific activity, so the need for oxygen is not critical.
Medical Lenses: They can be worn all day long and, in some cases, even overnight, which makes oxygen permeability vital to avoid complications such as corneal hypoxia.

### 5 Consequences of Oxygen Deprivation:

Recreational Lenses: If a recreational lens impedes oxygen passage, the consequences may be minor if worn for a short period of time.
Medical Lenses: Lack of oxygen can cause serious eye health problems, such as infection, inflammation and irreversible corneal damage.

To sum up; the main difference lies in the critical need for oxygen in medical lenses due to their extended wear and therapeutic function, while recreational lenses may not have the same requirements attending to their temporary use and different purposes. This highlights the importance of designing medical lenses that are safe and effective at maintaining ocular health.

### Selection of Material Permeability:

Dk/t is a measurement used to describe the oxygen permeability of contact lenses, especially gas permeable lenses. Dk is a value that indicates how well a material allows oxygen to pass through it. The higher this number, the better the oxygen permeability. The term ‘t’ refers to the thickness of the lens in millimeters.

So, when calculating the Dk/t, the amount of oxygen that can reach the surface of the eye through the lens is evaluated. A higher Dk/t means that the lens allows more oxygen, which is important for eye health.

Thus, Dk/t is a way of measuring how well a contact lens allows oxygen to pass through, considering both the material and the thickness of the lens.

Gas permeable (GP) contact lenses are primarily made of plastic materials such as silicone and acrylate. These materials allow oxygen to pass through them, which is vital for eye health. The structure of these polymers creates spaces that facilitate the passage of oxygen; unlike ophthalmic lenses, which are solid-structured materials that have no permeability.

Thanks to their design, GP lenses not only allow good oxygen circulation, but also provide clear vision and are less prone to accumulate debris. For these reasons, they are a popular choice for those seeking comfort and eye health.

The choice of gas permeable-such as oxygen-lenses is based on existing and in-use FDA-approved contact lens materials, that allow oxygen to pass through to the cornea. The final decision rests with the eye care professional, who evaluates the patients' individual needs, considering their ocular health and lifestyle. In addition, the shape of the eye and the patient's comfort are considered. Follow-up is performed to assess comfort and ocular health, allowing for adjustments if necessary. In sum, although there are regulated materials, lens selection depends on the practitioner's judgment and the patient's specific needs.

### Choice of Filling Solutions:

The choice of contact lens solutions that stay on the eye is generally based on 0.9% isotonic solutions, which are the safest and provide the necessary nutrients for ocular health. However, settlement selection may vary according to the individual patient's needs and is always subject to the judgment of eye care professionals. This judgment is based on the professionals' experience in the field, considering factors such as the length of time the optical device has been in use, and the patient's ocular health, comfort and specificity of the situation. This ensures that the most appropriate solution is chosen for each case casuistry.

### Liquid Chamber Solution:

9% saline is ideal for contact lenses or filling devices for several reasons:
1. proper osmolarity: 9% saline is formulated to have an osmolarity that resembles the natural eye fluid, which helps maintain ocular comfort and health.
2. Compatibility: This solution is generally well tolerated by ocular tissues and minimizes the risk of irritation or allergic reaction, all this compared to other solutions that may contain preservatives or additional ingredients.
3. Hydration: Thanks to its isotonic faculty, it helps to maintain hydration of the ocular surface, preventing dehydration and ensuring better visibility and comfort during use.
4. Infection prevention: The 9% solution can help create a less prone environment to the proliferation of bacteria and other pathogens, thus maintaining ocular health.
5. Ease of use: The simplicity of this solution makes it easy to use and apply, which is important for people who wear fillers and contact lenses.

In summary, the 9% saline solution offers a perfect balance of features that promote eye health and comfort for filling or contact lenses, while other solutions may not provide the same benefits.

The choice of solutions for filling contact lenses that stay on the eye is generally based on 0.9% isotonic solutions, which are the safest and provide the necessary nutrients for ocular health, as they have detergent preservatives avoid health vulnerabilities.

However, the election of solutions may vary according to the individual needs of the patient and is always subject to the criteria of eye care professionals. This judgment is based on the eye care professional's experience in the field, considering factors such as the patient's eye health, comfort and the specifics of the situation. This ensures that the most appropriate solution is chosen for each casuistry

There are several reasons why other solutions different to 9% saline serum cannot be used for contact lenses:
1. Inadequate osmolarity: other solutions may have an osmolarity which does not match with natural tears. This can cause irritation, dryness or discomfort when wearing the lenses.
2. Presence of preservatives: Many contact-lens cleanings and storage solutions contain preservatives that can irritate the eyes or cause allergic reactions. These preservatives can damage both the ocular surface and the lenses themselves.
3. Chemical composition: Some solutions may contain ingredients that are not safe for eyes or may interfere with the effectiveness of contact lenses, such as detergents or cleaning agents which can cause irritation.
4. Inappropriate PH: The PH of solutions is crucial to eye health. Other solutions may have a pH that is not compatible with eyes. This can cause discomfort and damage to the ocular surface.
5. Insufficient hydration: Some solutions may not provide the necessary hydration to keep contact lenses in optimal condition, which can result in blurred vision or the sensation of dry eyes.
6. Risk of infection: Using inadequate solutions can increase the risk of eye infections, as not all solutions offer the antimicrobial properties needed to protect the eyes.

For these reasons, it is essential to use solutions specifically designed for contact lenses, such as 9% saline, to ensure comfort, eye health and lens efficacy.

The subject disclosure provides a method of treating ocular conditions. By way of example, a method treating keratoconus may include the following step. First the patient's corneal topography is measured and other ocular properties such as interpupillary distance and the measurements mentioned above, as well as measurements of the patient's refractive error (by way of a retinoscope), assessment of preceptive status, and the like.

Upon an initial assessment of keratoconus, the patient is fitted with the subject matter goggles. Then the cavities are filled, through the auxiliary inlet 3, with isotonic solution and/or immersive solution having approximately 9% saline solution.

Then the patient is tested for anti-positives and another vision tests is applied based on the radius of curvature of an initial set of lens in the goggle. It is understood that the sagittal height of the lens may be more than two mm, and because the peripheral edge of the lens 1 may engage the face of the goggles so that the sagittal height protrudes almost entirely beyond the face plate, the sagittal height adds volume to the cavity of the google frame's cavity, and this additional volume of immersive solution that occupies the volume adjacent the lens 1 requires the practitioner to reassess the amount of treatment solution that is to be added to the immersive solution.

With the goggles on, the practitioner measures the pano-scopic angle with a measure panoramic mirror (from above the patient), which measures the angle of curvature of the goggle relative to the face of the patient. A panoramic triangle may be identified, which may assist in ensuring the goggles are straight relative to the patient's face.

From this, a final radius of curvature is determined for the lens 1 based on the dioptric power needed in view of initial radius of curvature (typically approximately 8.6 mm) and the initial isotonic/saline immersive solution. Then from this final radius of curvature, then a final formulation of immersive solution with medicament that treats keratoconus, such as copper sulfate solutions. The percentage of, in this example, copper sulfate is a function of the overall volume of the relative cavity (were that overall volume is a function of the final radius of curvature). Thereby, through this iterative process of first establishing the final radius of curvature based on the re-assessment of the patient wearing the initial lens with a cavity sealed to their skin, wherein the cavity is filled with an isotonic immersive solution.

It should be noted that the subject disclosure may be embodied in a single patch solution, wherein the peripheral edge of the patch provides adherent that forms a seal around the skin of one eye, whereby a sealed cavity is formed for retaining immersive solution, while an central portion of the patch provides a gasket of the like for retaining a systemic lens whereby substantially all of that lens's sagittal height protrudes beyond the patch.

As used in this application, the term “about” or “approximately” refers to a range of values within plus or minus 10% of the specified number. And the term “substantially” refers to up to 80% or more of an entirety. Recitation of ranges of values herein are not intended to be limiting, referring instead individually to any and all values falling within the range, unless otherwise indicated, and each separate value within such a range is incorporated into the specification as if it were individually recited herein.

For purposes of this disclosure, the term “aligned” means parallel, substantially parallel, or forming an angle of less than 35.0 degrees. For purposes of this disclosure, the term “transverse” means perpendicular, substantially perpendicular, or forming an angle between 55.0 and 125.0 degrees. Also, for purposes of this disclosure, the term “length” means the longest dimension of an object. Also, for purposes of this disclosure, the term “width” means the dimension of an object from side to side. For the purposes of this disclosure, the term “above” generally means superjacent, substantially superjacent, or higher than another object although not directly overlying the object. Further, for purposes of this disclosure, the term “mechanical communication” generally refers to components being in direct physical contact with each other or being in indirect physical contact with each other where movement of one component affect the position of the other.

The use of any and all examples, or exemplary language (“e.g.,” “such as,” or the like) provided herein, is intended merely to better illuminate the embodiments and does not pose a limitation on the scope of the embodiments or the claims. No language in the specification should be construed as indicating any unclaimed element as essential to the practice of the disclosed embodiments.

In the following description, it is understood that terms such as “first,” “second,” “top,” “bottom,” “up,” “down,” and the like, are words of convenience and are not to be construed as limiting terms unless specifically stated to the contrary.

It should be understood, of course, that the foregoing relates to exemplary embodiments of the invention and that modifications may be made without departing from the spirit and scope of the invention as set forth in the following claims.

What is claimed is:

1. A method of treating an ocular condition that adversely affects the vision of a patient, the method comprising:
sealing a mounting frame against the patient so as to define a sealed chamber that circumscribes an eye of the patient, wherein the mounting frame provides a corrective lens for correcting the vision of said eye;
determining a vertex distance, a base curve, and the gas permeability for the corrective lens based on a diopter of the eye; and
selectively filling the chamber with a medical solution based on a treatment plan for the ocular condition, wherein the treatment plan includes a measurement of the frame.

2. A method of treating a medical condition of a patient with an immersive solution having a medicament to remedy the medical condition, the method comprising:
assessing a set of ocular properties of the patient;
sealing a cavity around an eye of the patient by way of a frame with a lens holder disposed in a forward-facing element of the frame, wherein the lens holder retains a first lens having a first radius of curvature;
filling the cavity with an isotonic solution;
re-assessing the set of ocular properties of the patient with the first lens and the isotonic solution disposed in a field of view of the patient;
determining a second lens with a second radius of curvature; and
determining a percentage or weight of the medicament to be mixed with the isotonic solution to form the immersive solution within the cavity, based on the second radius of curvature and the re-assessed set of ocular properties.
